(19) United States
(12) Patent Application Publication
(10) Pub. No.: US 2021/0161941 A1
(43) Pub. Date: Jun. 3, 2021

(54) Formulation for the Treatment of Polymorphisms in Methyl Metabolizing Genes and Methods of Treatment Thereof

(71) Applicant: Gay B. Ben Tre, Providence, RI (US)

(72) Inventor: Gay B. Ben Tre, Providence, RI (US)

(21) Appl. No.: 16/960,874

(22) PCT Filed: Jan. 25, 2019
(86) PCT No.: PCT/US2019/015082

§ 371 Date: Jul. 8, 2020

Related U.S. Application Data
(60) Provisional application No. 62/796,170, filed on Jan. 24, 2019.
(60) Provisional application No. 62/622,850, filed on Jan. 27, 2018.

(51) Int. Cl. A61K 31/714; A61K 31/519; A61K 31/675; A61K 31/19; A61K 45/06; A61P 43/00; A23L 33/15; A23L 33/12; A23L 33/00

(52) U.S. Cl. CPC A61K 31/714; A61K 31/519; A61K 31/675; A61K 31/19; A23V 2002/00; A61P 43/00; A23L 33/15; A23L 33/12; A23L 33/40; A61K 45/06

(57) ABSTRACT

Pharmaceutical compositions comprised of a combination of three bioactive forms of vitamins: 5-methyltetrahydrofolate (methylated folate or vitamin B9), methylcobalamin (methylated B12), and pyridoxal 5-phosphate (B6), which may be supplemented with butyrate, for the promotion of normal child development and healthy functioning in patients with polymorphisms within genes required for the correct metabolism of the unmodified versions of the above vitamins, wherein the patient is either under the age of 12 or a pregnant or nursing mother.

### ASSERTION OF PRIORITY

This application claims priority to U.S. Application No. 62/622,850, filed Jan. 27, 2018, the entire contents of which is incorporated herein by reference.

### FIELD OF THE INVENTION

The invention relates to a combination of three bioactive forms of vitamins: 5-methyltetrahydrofolate (methylated folate or vitamin B9), methylcobalamin (methylated B12), and pyridoxal 5-phosphate (B6), which may be supplemented with butyrate, for the promotion of normal child development and healthy functioning in patients with polymorphisms within genes required for the correct metabolism of the unmodified versions of the above vitamins, wherein the patient is either under the age of 12 or a pregnant or nursing mother. The invention further relates to a dosing schedule designed to promote health in the above patient population.

### BACKGROUND

Proper intake of B vitamins such as folate (B9) is essential for maintaining health due to the role that B vitamins play as cofactors in many pathways of cell metabolism and DNA repair. B vitamin deficiency has been implicated in numerous diseases, including anemia, birth defects in pregnant mothers, and behavioral and neurological disorders. In particular, folate deficiency results in a lack of adequate DNA methylation, an epigenetic modification consisting of adding a methyl group to the 5-carbon position of cysteine, primarily within cysteine-guanine dinucleotides. DNA methylation is necessary for proper regulation of gene expression and chromosome integrity. The link between methylating nutrients such as B vitamins and diseases caused by impaired DNA methylation has been well established (Glier M B, Green T J, and Devlin A M, (2014) Mol Nutr Food Res., 58(1):172-82, Kok D E et al., (2015) Clin Epigenetics, 14(7):121, and Pusceddu I et al., (2016), Eur J Nutr., 55(5):1863-73). Additionally, supplementation with folate in a pregnant mother has been shown to promote DNA methylation in the child (Paparo L et al., (2014) Nutrients, 6:4706-4719.). While folate is required for DNA methylation, Vitamin B6 and Vitamin B12 have now also been identified as critical co-factors.

B12 is in demand not only for folate metabolism and the methylation pathway but for the production of red blood cells (Greer J P (2014). Wintrobe's Clinical Hematology Thirteenth Edition) and myelin sheathing of the nerves and maintenance of the nervous system (Miller A, et al., (2005), J Neuro Sci., 233(1-2):93-7.). Vitamin B6 is one of the most in demand nutrients of all and plays an important role in red blood cell production, brain development during gestation and infancy, immune function, heavy metal detox, hormone and neurotransmitter production, and over 100 enzymatic reactions in metabolic pathways. (Combs, G. F. (2007) The Vitamins: Fundamental Aspects in Nutrition and Health (3rd ed.)) Competition for bioavailable nutrients such as B12 and B6 can result in a systemic shortage which may negatively impact methylation, and thus, gene expression.

While most B vitamin deficiencies can be easily treated by supplementation, there is a significant percentage of the population who are unable to properly utilize unmodified forms of these molecules due to mutations in methyl metabolizing genes, such as MTHFR (Guéant-Rodriguez R M, et al., (2006) Am J Clin Nutr, 83:701-7). This gene encodes the enzyme methylenetetrahydrofolate reductase, which irreversibly reduces 5,10-methylenetetrahydrofolate to 5-methyltetrahydrofolate which, in turn, is necessary to convert homocysteine (a potentially toxic amino acid) to methionine by the enzyme methionine synthase. Patients with common SNPs (single nucleotide polymorphisms) in the MTHFR gene, such as C677T, have impaired metabolism of folate (Reilly R et al., (2014) Proc Nutr Soc., 73(1):47-56). These polymorphisms have been implicated in numerous disorders, particularly developmental disorders such as cleft palate, spina bifida, and anencephaly. Conventional B vitamin supplementation in patients with MTHFR^{C677T} is therefore insufficient and possibly dangerous due to the impairment of this metabolic pathway.

In response to this need, modified forms of methylated folate have been developed and patented to date: Metafolin™ (U.S. Pat. No. 7,560,123) by Merck and Quatrefolic™ (application PCT/IB2015/060027) by Gnosis. There are also generic forms of methylated folate available for supplement manufacturing. While methylated folate can compensate for polymorphisms in MTHFR, mutations in any of the other genes requiring bioactive B6 and B12 can interfere with adequate methylation of DNA and other essential cell processes. Thus far, no manufacturer is expressly addressing the need for the bioactive forms of all three of the B vitamins in order to maximize compensation for polymorphisms in the cluster of genes of the one carbon metabolism pathway (MTHFR, MTR, MTRR, MTHFD1, and SHMT).

Existing B vitamin supplements currently on the market also are often prescribed with dosing schedules in excess of what is necessary to maintain a healthy adult and may contain additional ingredients whose safety has not been tested on children, pregnant, or nursing mothers. Furthermore, excess supplementation with folic acid inhibits normal functions of the MTHFR protein, creating similar problems as MTHFR polymorphisms (Christensen K. et al., (2015) Am J Clin Nutr, 101:646-58). Excess circulating folic acid is potentially dangerous. (Wiens, D. and DeSoto, M C, (2017) Brain Sci., 7(11): 149, and Sauer J. et al., (2009) Curr Opin Clin Nutr Metab Care. 12(1): 30-36.) Because folic acid requires reduction to dihydrofolate and then to tetrahydrofolate before it can enter the folate cycle, those with SNPs in the MTHFR gene are especially vulnerable to toxicity resulting from excess circulating folic acid.

There is a need for a safer method of supplementation of folate and one-carbon metabolism cofactors in deficient patients twelve years of age and under, pregnant, or nursing mothers, and prospective parents that also have polymorphisms in one or more genes involved in the one carbon metabolism (biological processes for DNA synthesis, repair and other methylation reactions) pathway.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

Within the framework of the present description and in the subsequent claims, except where otherwise indicated, all numbers expressing amounts, quantities, percentages, and so forth, are to be understood as being preceded in all instances by the term “about”. As used herein, the term “about” is defined as ±5%. Also, all ranges of numerical entities include all the possible combinations of the maximum and minimum numerical values and all the possible intermediate ranges therein, in addition to those specifically indicated hereafter.

The term “and/or” as used herein is defined as the possibility of having one or the other or both. For example, “A and/or B” provides for the scenarios of having just A or just B or a combination of A and B. If the claim reads A and/or B and/or C, the composition may include A alone, B alone, C alone, A and B but not C, B and C but not A, A and C but not B or all three A, B and C as components.

The term “active form” herein refers to the metabolite form of the inactive prodrug that is metabolized within the body into its active form, regardless of the source of said prodrug.

The term “pharmaceutically acceptable salts or derivatives” herein refers to those salts or derivatives which possess the biological effectiveness and properties of the salified or derivatized compound and which do not produce adverse reactions when administered to a mammal, preferably a human. The pharmaceutically acceptable salts may be inorganic or organic salts; examples of pharmaceutically acceptable salts include but are not limited to: carbonate, hydrochloride, hydrobromide, sulphate; hydrogen sulphate; citrate, maleate, fumarate, tifluoroacetate, 2-naphthalenesulphonate, and para-toluenesulphonate. Further information on pharmaceutically acceptable salts can be found in Handbook of pharmaceutical salts, P. Stahl, C. Wermuth, WILEY-VCH, 127-133, 2008, herein incorporated by reference. The pharmaceutically acceptable derivatives include the esters, the ethers and the N-oxides.

The term “physiologically acceptable excipient” herein refers to a substance devoid of any pharmacological effect of its own and which does not produce adverse reactions when administered to a mammal, preferably a human. Physiologically acceptable excipients are well known in the art and are disclosed, for instance in the Handbook of Pharmaceutical Excipients, sixth edition 2009, herein incorporated by reference.

### SUMMARY

The invention is considered to be a holistic formula composed of maximally bioavailable and non-toxic vitamins uniquely suited for use by consumers with compromised methylation due to SNPs of one or more methylation genes, including MTHFR, MTR, MTRR, MTHFD1, and SHMT. These same vitamins can also safely be taken if there are no SNPs or if the methylation status of the individual consumer is unknown.

One carbon metabolism, a biological process for DNA synthesis, repair and other methylation reactions, must be supported by sufficient quantities of bioavailable dietary folate (B9), and bioavailable B12 and B6, which act as co-factors. While there is no direct chemical synergy between the composition vitamins, when the genetically compromised methylation cycle is supplemented with them, there is not only a cumulative effect on the output of methyl molecules, but there is a synergistic effect on the DNA, RNA and chromatin of each compromised methylation gene, insuring normalization of function and physiology. (Crider, K S. et al., (2012) Adv Nutr. 3(1):21-38) A shortage of any of the three; B9, B6 and B12, will upset this metabolic pathway. This has been shown to have profoundly disruptive effects on the health of the pregnant mother, and on cell proliferation, growth and function in the fetus, the neonate and the developing child. (Glier M B, Green T J, and Devlin A M, (2014) Mol Nutr Food Res., 58(1):172-82, and Furness D, et al., (2013) Matern Child Nutr., 9(2):155-66.) Existing folate and folic acid supplements, including those supplements also providing B12, are inadequate for the task; B6 intake must also be supplemented.

The prior art on the subject of B vitamin deficiency focuses on the increase in homocysteine resulting from insufficient methylation as the contributing factor to congenital defects in the pediatric population. The inventor of the instant application discovered that the congenital defects and developmental delays, however, are the result of a shortage of bioavailable folate, B6, and B12 needed for production and activities of DNA, RNA, and chromatin which regulate normal development, and for the growth and subsequent metabolic activity and functioning of the newly created and differentiated cells and tissues (Kalani A, et al., (2014) J Mol Neurosci., 52(2):202-215). In the case of developmental delays, the claimed invention provides the nutrients necessary to overcome the delays, thereby eliminating symptoms of the delays. The invention is focused on prevention, rather than curing a homocysteine-driven disease.

The instant invention further includes the use of short chain fatty acids and their conjugate bases, in particular butyrate, as an additional promoter of epigenetic health. Supplementation with butyrate has been demonstrated to have many benefits to both neurological, intestinal, and epigenetic health, possibly due to butyrate's role as a histone deacetylase inhibitor, and butyrate has been suggested as a treatment for several developmental diseases. (Canani R B, DiCostanzo M, and Leone L, (2014) Clin Epigenet, 4:4, and Ong T P, Moreno F S, and Ross S A (2011) J Nutrigenet Nutrigenomics, 4:275-292.) In the context of the invention, butyrate exerts a synergistic effect on epigenetic health with the bioactive B vitamins of the invention.

Butyrate has also demonstrated an anti-inflammatory effect by the suppression of NF-κ-B activation, inhibition of interferon gamma production, and upregulation of peroxisome proliferator-activated receptor gamma. (Canani R B, DiCostanzo M, and Leone L, (2014) Clin Epigenet, 4:4.) This property of butyrate is further utilized in the instant invention in the prevention of neurodegenerative disorders by promoting healthy DNA methylation in utero. (Richetto J et al., (2017) Biol Psychiatry, 81(3):265-276.) Similarly, butyrate has been shown to be effective in the treatment of neonatal hypoxic-ischemic (HI) injury, possibly due to its activity as a histone deacetylase inhibitor (Ziemka-Nalecz M, et al., (2017) Mol Neurobiol., 54(7):5300-5318). The instant invention further utilizes the histone deacetylase inhibiting property of butyrate to provide additional neuroprotective effects.

The invention is for the promotion of normal child development and healthy functioning. Reproductive events and child development will occur in the complex and orderly way nature intended and the imbalances in functioning that result in infertility, miscarriage, premature birth, congenital defects, and developmental delays will be prevented. The neurologic developmental problems of childhood are prevented, and the child is supported in reaching his/her potential. In addition, by stabilizing physiological processes with supplementation, cardiovascular, metabolic, neurologic, etc. disease states have a decreased likelihood of occurring later in life.

Adequate supplies of absorbable B vitamins raise the bar for adaptability and insure resilience to physiological, mental and psychological challenges and stressors. This is a critical edge throughout life. Bioavailable vitamins B6, B9, and B12 not only drive one carbon metabolism, but also support the production of antioxidants and Phase I, Phase II, and Phase III detoxification. Symptoms arising from free radical production and the inability to detox the system may be prevented without supplementing any other substances that frequently cause an over-correction of the problem and upset redox homeostasis. Likewise, proper functionality of the one carbon metabolism pathway is necessary for proper epigenetic health by promoting correct DNA methylation. (Bae S, et al., (2014) Epigenetics, 9:(3)396-403.) The addition of butyrate in the instant invention is designed to improve the efficacy of the bioactive vitamins in promoting epigenetic health (Lu R, et al., (2008) Epigenetics, 3(6):330-5).

Daily dosing of the composition B vitamins is required because, as water soluble molecules, the body is unable to store them, in contrast to Vitamin A, D, etc. The invention is specifically targeted to meet the increased demands for nutrients by the pediatric population to insure normal growth and healthy development. The invention will also provide support for the prospective parents who will be contributing the egg and sperm, chromosomes and epigenetic information. After conception, the invention will supply the needs of the growing embryo and fetus by supplementing the mother. Postnatally, the infant can be supplemented independently of the mother, regardless of a breast milk or formula diet. The invention is intended for continued use as the child grows, with varying dosage schedules.

The instant invention improves over existing B vitamin supplements known in the art in four ways. First, the invention comprises only bioactive forms of folate, B6, and B12. Second, the invention lacks other additives not essential to the proper maintenance of the one carbon metabolism pathway, such as betaine, zinc, or cysteine derivatives. Third, the invention prescribes a dosing schedule that is designed to be safer in pregnant and nursing mothers, as well as children under the age of twelve. Fourth, the invention further comprises butyrate, the conjugate base of the short chain fatty acid butyric acid (C₄H₇O₂—), to facilitate absorption of the bioactive B vitamins present and provide additional support for maintenance of epigenetic health.

The present invention therefore is a combination of the bioactive forms of folate, vitamin B6 and B12, alone or together with butyrate, designed to meet the metabolic needs of pregnant or nursing mothers, or children under the age of twelve who would otherwise not fully benefit from conventional B vitamin supplements due to their genetic backgrounds.

### DETAILED DESCRIPTION OF THE INVENTION

The instant invention is directed to a supplement formulation of bioactive versions of folate, vitamin B6, and vitamin B12 for a patient in need of such supplementation but unable to properly metabolize the unmodified versions of the above vitamins due to SNPs in genes of the one carbon metabolism pathway. Specifically, these unmodified vitamins are modified as follows: folate is methylated at the fifth carbon to produce 5-methyltetrahydrofolate, pyridoxine (unmodified B6) is phosphorylated into pyridoxal-5-phosphate, and cobalamin (unmodified B12) is methylated at the cobalt to produce methylcobalamin. The combination of these three bioactive vitamins is sold under the trade name “EPIFOLIN”.

In a preferred embodiment of the claimed invention, butyrate is co-administered with the vitamins of EPIFOLIN to the patient in need of treatment. The combination of the three bioactive vitamins mentioned above with butyrate is sold under the trade name “EPIFOLIN+”.

Another embodiment of the invention is directed to a method for the promotion of normal child development and healthy functioning in patients with polymorphisms within genes comprising administering to a subject a therapeutically beneficial amount of EPIFOLIN (5-methyltetrahydrofolate (methylated folate or vitamin B9), methylcobalamin (methylated B12), and pyridoxal 5-phosphate (B6)) or EPIFOLIN+(5-methyltetrahydrofolate (methylated folate or vitamin B9), methylcobalamin (methylated B12), and pyridoxal 5-phosphate (B6), and butyrate)) for the maintenance or promotion of normal physiological development and function in a patient in need of supplementation with the above vitamins, according to the following dosage schedule:

5-methyltetrahydrofolate and/or the pharmaceutically acceptable salts thereof in an amount comprised as follows:

Newborn to 1 yrs.: Between 22 to 150 mcg, preferably 100 mcg.

1 yr. to 3 yrs.: Between 49 to 300 mcg, preferably 200 mcg.

4 yrs. To 8 yrs.: Between 132 mcg to 600 mcg, preferably 400 mcg.

9 yrs. To 12 yrs.: Between 132 mcg to 600 mcg, preferably 400 mcg.

Preconception, pre-natal, breastfeeding: Between 198 mcg to 1,000 mcg, preferably 800 mcg.

Methylcobalamin and/or the pharmaceutically acceptable salts thereof in an amount comprised as follows:

Newborn to 1 yr.: Between 0.16 to 20 mcg, preferably 0.5 mcg.

1 yr. to 3 yrs.: Between 0.3 to 40 mcg, preferably 1.0 mcg.

4 yrs. To 8 yrs.: Between 0.8 to 2,000 mcg, preferably 2.0 mcg.

9 yrs. To 12 yrs.: Between 0.8 to 2,000 mcg, preferably 2.4 mcg.

Preconception, pre-natal, breastfeeding: Between 0.92 mcg to 10,000 mcg, preferably 350 mcg.

Pyridoxyl-5-phosphate and/or a pharmaceutically acceptable salt thereof in an amount comprised as follows:

Newborn to 1 yr.: Between 0.1 mg to 3.6 mg, preferably 1.8 mg.

1 yr. to 3 yrs.: Between 0.16 mg to 30 mg, preferably 3.6 mg.

4 yrs. To 8 yrs.: Between 0.56 mg to 40 mg, preferably 7.2 mg.

9 yrs. To 12 yrs.: Between 0.56 mg to 40 mg, preferably 7.2 mg.

Preconception, pre-natal, breastfeeding: Between 0.66 mg to 100.0 mg, preferably 30.0 mg.

Butyrate and/or a pharmaceutically acceptable salt thereof in an amount comprised as follows:

Newborn to 1 yr.: Between 2.0 mg to 200 mg, preferably 100.0 mg.

1 yr. to 3 yrs.: Between 4.0 mg to 300 mg, preferably 200.0 mg.

4 yrs. To 8 yrs.: Between 6.0 mg to 500 mg, preferably 400.0 mg.

9 yrs. To 12 yrs.: Between 10.0 mg to 750 mg, preferably 400.0 mg.

Preconception, pre-natal, breastfeeding: Between 20.0 mg to 1,200.0 mg, preferably 200.0 mg.

In one embodiment of the invention, the subject is a pregnant mother. In a further embodiment, the method is directed to the prevention of birth defects of the mother's child, including but not limited to, neural tube defects such as spina bifida, anencephaly, hyperhomocystemia leading to loss of pregnancy, low birth weight, and congenital heart defects (Furness D, et al., (2013) Matern Child Nutr., 9(2):155-66 and Memon S and Pratten M K, (2013) Repor. Toxicol., 35:117-24). In a further embodiment, the subject is a nursing mother.

In another embodiment of the invention, EPIFOLIN and EPIFOLIN+ are administered as a powder for oral consumption. In a further embodiment, the powder is contained within a capsule for ease of oral consumption. In other embodiments, the bioactive vitamins and co-factors of EPIFOLIN and EPIFOLIN+ are combined with common pharmaceutical binding agents to prepare the invention for consumption as a pill or as an elixir.

In other embodiments, EPIFOLIN and EPIFOLIN+ are administered as an injection, suppository, or intravenously.

In a further embodiment of the invention, EPIFOLIN and EPIFOLIN+ are administered with a physiologically acceptable excipient, selected from the group comprising bulking agents, mixing agents, anticaking agents, aggregating agents or binders, lubricants, coating agents, stabilizers, natural flavorings, or a mixture thereof, preferably bulking agents, anticaking agents, stabilizers and natural flavorings and a mixture thereof.

In yet a further embodiment, EPIFOLIN and EPIFOLIN+ are administered with a physiologically acceptable surfactant. A “surfactant” as used herein is any compound that can greatly reduce the surface tension of water when used in very low concentrations.

In yet another further embodiment, EPIFOLIN and EPIFOLIN+ are administered with a physiologically acceptable buffer. A “buffer” as used herein is any acid or salt combination which is pharmaceutically acceptable and capable of maintaining the composition of the present invention within a desired pH range.

In yet another further embodiment, EPIFOLIN and EPIFOLIN+ are administered with a physiologically acceptable preservative. As used herein, a preservative is pharmaceutically acceptable, suitable for administration to a subject, which inhibits, prevents or delays the growth or microorganisms including, for example bacteria, viruses and fungi in the compositions of the present invention. Suitable preservatives for use in the compositions and methods of the present invention include, but are not limited to, cresols, benzyl alcohol, phenol, benzalkonium chloride, benzethonium chloride, chlorobutanol, phenylethyl alcohol, methyl paraben, propyl paraben, thiomersal and phenylmercuric nitrate and acetate, citric acid, sodium citrate, potassium sorbate, vitamin C (ascorbic acid), sodium ascorbate, sodium benzoate, potassium benzoate, grapefruit seed oil, or vegetable glycerin. In one embodiment, the preservative is m-cresol, chlorocresol or phenol.

In an embodiment of the invention, EPIFOLIN and EPIFOLIN+ are administered as a food additive or dietary supplement.

In an embodiment of the invention, the method is used as pregnancy support and/or as perinatal support and/or as fertility enhancer for prospective mothers and fathers seeking parenthood by natural conception or with assisted reproductive technologies.

In an embodiment of the invention, the method is used for stimulating antioxidant production and Phase I, Phase II, and Phase III detoxification, preferably in the pediatric population, and in the preconception and pregnant population.

In an embodiment of the invention, the method is used for supplementing the culture medium used for in vitro maturation of ex vivo oocytes as well as embryos obtained from naturally matured oocytes, as well as stem cells.

In various embodiments of the invention, the method is used for the prevention of and/or treatment of the following diseases and conditions:
Failure to Thrive, Failure to Thrive Syndrome, and in the prevention and/or treatment of certain early pediatric eating disorders, preferably in the pediatric population, and in the breastfeeding population.
Abnormal growth and development, especially in the prevention and/or treatment of decelerated or arrested physical growth, in the prevention and/or treatment of delayed closure or incomplete ossification of the fontanelles, in the prevention and/or treatment of delayed, asymmetrical, and/or underdevelopment of the facial bones including jaw and teeth, preferably in the pediatric population, also in the breastfeeding population.
Delayed repair of injuries to the bones and/or epiphyseal plates (growth plates), preferably in the pediatric population.
Abnormal or delayed growth and development, especially in the prevention and/or treatment of delayed speech and language acquisition, and Pervasive Developmental Disorder (PDD), preferably in the pediatric population.
Autism Spectrum Disorders (ASD), Delayed Neurological Development Syndromes, Pediatric Autoimmune Neuropsychiatric Disorders Associated with Streptococcal Infections (PANDAS), and generalized anxiety and panic disorder, preferably in the pediatric population.
Attention Deficit Disorder (ADD), Attention Deficit Hyperactivity Disorder (ADHD), Obsessive Compulsive Disorder (OCD), and Deficits in Attention, Motor control and Perception (DAMP), preferably in the pediatric population.
Placental deficiencies including placenta previa and placental abruption, preferably in the pregnant population.
Congenital malformations and deformations of the tongue, mouth and pharynx of the developing embryo and fetus, including cleft lip and palate, ankyloglossia, macroglossia, and hypoglossia, preferably in the pregnant population.
Congenital malformations and deformations of the esophagus and stomach in the developing embryo and fetus, including esophageal atresia, tracheoesophageal fistula, esophageal web and Schatzki ring, pyloric stenosis and hiatus hernia, preferably in the pregnant population.
Malformations and deformations of the intestines of the developing embryo and fetus, including intestinal atresia, duodenal atresia, Merckel's diverticulum, Hirschsprung's disease, intestinal malrotation, dolichocolon, and enteric duplication cyst, preferably in the pregnant population.
Malformations and deformations of the rectum and anal canal of the developing embryo and fetus, including imperforate anus, rectovestibular fistula, persistent cloaca, and rectal atresia, preferably in the pregnant population.
Malformations and deformations of the pancreas in the developing embryo and fetus, including annular pancreas, accessory pancreas, Johanson-Blizzard syndrome, and pancreas divisum, preferably in the pregnant population.
Malformations and deformations of the bile ducts and liver of the developing embryo and fetus, including choledochal cysts, Caroli disease, biliary atresia, Alagille syndrome and polycystic liver disease, preferably in the pregnant population.
Congenital malformation and deformations of the neural tube, brain, and spine of the developing embryo and fetus, including spina bifida, anencephaly, encephalocele, hydrocephalus, and facial clefts, preferably in the pregnant population.
Malformation and deformations of the heart and heart vessels of the developing embryo and fetus, including tetralogy of Fallot, ventricular septal defect, atrial septal defect, and patent ductus arteriosus, preferably in the pregnant population.
Malformation and deformations of the renal and urinary tract of the developing embryo and fetus, including hypospadias, double ureter, obstructive defects of the renal pelvis, renal agenesis, and renal dysplasia, preferably in the pregnant population.
Congenital malformation and deformations of the bones of the developing embryo and fetus, including facial asymmetry, congenital limb amputations, limb hypoplasia, and limb deficiencies of the developing embryo and fetus, preferably in the pregnant population.
Prenatal toxicity resulting in teratogenesis and characterized by structural or functional defects in the developing embryo or fetus, preferably in the pregnant population.
Spontaneous abortion, miscarriage, preeclampsia, and birth complications including premature birth, preferably in the pregnant population.
Complications of pregnancy and birth that result in injury to the mother and maternal mortality, preferably in the pregnant population.
First trimester identical (monozygotic) twin loss and prevention of conjoined twins, preferably in the pregnant population.
Lasting neurological dysfunction resulting from neonatal hypoxic/ischemic (HI) injury, preferably in the neonatal and infant population.
Low concentrations of oxygen and nutrients in the blood, with resulting teratogenesis, developmental disruption, neurological and organ damage and growth retardation in the fetus due to maternal exposure to environmental toxins, preferably in the pregnant population.
Low concentrations of oxygen and nutrients in the blood, resulting in developmental disruption, neurological damage, organ damage, and growth retardation in the infant and child resulting from exposure to environmental toxins, preferably in the pediatric population.
The effects of environmental toxin exposure resulting in inhibited and/or decreased DNA and RNA repair, and detoxifying and oxidative stress genes, preferably in the pediatric population, and the preconception, pregnant and breastfeeding population.
Cellular damage to lung and bronchial tissues resulting from exposure to environmental toxins, preferably in the pediatric population, and the preconception, pregnant and breastfeeding population.
Low concentrations of oxygen and nutrients in the blood resulting in epigenetic modifications, impaired brain function, and developmental retardation resulting from exposure to environmental toxins, preferably in the pediatric population, and the preconception, pregnant and breastfeeding population.
Vaccinosis and vaccination toxicity, preferably in the pediatric population.
Attention Deficit Disorder (ADD), Attention Deficit Hyperactivity Disorder (ADHD), and Obsessive-Compulsive Disorder (OCD), and generalized anxiety and panic disorders, preferably in the pediatric population, and in the preconception, pregnant and breastfeeding population.
Increased and dysregulated catechol-O-methyltransferase levels in the brain resulting from genetic polymorphisms (SNPs) of the MTHFR, MTR, MTRR, MTHFD1, and SHMT and COMT genes, preferably in the pediatric population, and in the preconception, pregnant and breastfeeding population.

In various embodiments of the invention, EPIFOLIN and EPIFOLIN+ are further supplemented with antioxidants and other compounds, as described below.

In an embodiment of the invention, EPIFOLIN and EPIFOLIN+ are used in the treatment of exposure to environmental pollutants, xenobiotics, or other harmful substances. In a further embodiment, the bioactive vitamins of EPIFOLIN and EPIFOLIN+ additionally contain one or more of the following: vitamin D3 (cholecalciferol), selenium, zinc, potassium citrate, magnesium citrate. manganese gluconate dihydrate, lutein, quercetin dihydrate or other dietary flavonoid, curcumin, turmeric (Curcuma longa), ashwaganda (Withania somnifera), milk thistle, the steroidal saponins, triterpenoid saponins and/or the biologically active compounds in these herbs.

In an embodiment of the invention, EPIFOLIN and EPIFOLIN+ are used in the treatment of anxiety due to traumatic stress and/or additional SNPs in the COMT and MAO genes. In a further embodiment, the bioactive vitamins of EPIFOLIN and EPIFOLIN+ additionally contain one or more of the following: L-theanine, GABA (gamma-aminobutyric acid), magnesium citrate, potassium citrate, quercetin dihydrate or other flavonoids, curcumin, turmeric (Curcuma longa), white peony (Paeoniae lactiflorae radix), schisandra (Schisandra Chinenesis fructus), Porea (Poriae Cocos sclerotium), lily bulb (LiIllium brownii bulb), red jujube dates (Zizyphi spinosis fructus) zizyphus (Zizyphi spinosi semen), licorice (Glycyrrhiza uralensis radix), the steroidal saponins, the triterpenoid saponins and/or the biologically active compounds in these herbs,

In an embodiment of the invention, EPIFOLIN and EPIFOLIN+ are used in the treatment of male infertility related to low sperm count and/or motility, poor sperm morphology and chromosomal abnormalities, and when Artificial Reproductive Technology is undertaken. In a further embodiment, the bioactive vitamins of EPIFOLIN and EPIFOLIN+ additionally contain one or more of the following: vitamin D3 (cholecalciferol), selenium, zinc, quercetin dihydrate or other dietary flavonoid, ashwaganda (Withania somnifera), Polygonum multiflorum root, Eucommia bark, Astragalus root, or Epimedium grandiflorum herba, the steroidal saponins, triterpenoid saponins, and/or the biologically active compounds in these herbs.

In an embodiment of the invention, EPIFOLIN and EPIFOLIN+ are used in the treatment of female infertility related to PCOS, age related fertility decline, hormone imbalance, toxic stress, DES syndrome, menstrual irregularity, failure to implant, frequent miscarriage, other chromosomal abnormalities, and when Artificial Reproductive Technology is undertaken. In a further embodiment, the bioactive vitamins of EPIFOLIN and EPIFOLIN+ additionally contain one or more of the following: vitamin D3 (cholecalciferol), choline, myo-inositol, quercetin dihydrate or other bioflavonoids, Vitex agnus-castus fruit, dong quai root, rehmannia root, Polygonum multiflorum root, liycium fruit, the steroidal saponins, triterpenoid saponins, and/or the biologically active compounds in these herbs.

In an embodiment of the invention, EPIFOLIN and EPIFOLIN+ are used in the treatment of cognitive deficit in children 4 years and older. In a further embodiment, the bioactive vitamins of EPIFOLIN and EPIFOLIN+ additionally contain one or more of the following: choline, lutein, quercetin dihydrate or other bioflavonoid, zizyphus (Zizyphi spinosi semen), Chinese wolfberry (Fructus lycium), Atractylodis macrocephalae rhizome, white peony (Paeoniae lactiflorae radix), Polygonum multiflorum root, Eucommia bark, vitex (Vitex agnus-castus) fruit, Platycodon grandifloras, the steroidal saponinds, triterpenoids saponins, and/or biologically active compounds in these herbs.

In an embodiment of the invention, EPIFOLIN and EPIFOLIN+ are used in the treatment of vaccinosis or disruption of folate metabolism due to exposure to vaccines. In a further embodiment, the bioactive vitamins of EPIFOLIN and EPIFOLIN+ additionally contain one or more of the following: potassium citrate, magnesium citrate.

### EXEMPLIFICATION

The invention is intended for the maintenance of health in people with a polymorphism in one or more genes in the one carbon metabolism pathway, which includes but is not limited to, MTHFR, MTR, MTRR, MTHFD1, and SHMT. The presence of these polymorphisms can be detected from conventional genetic profiles, obtained from buccal swabs. Short-term efficacy of the invention can be monitored by observing decreased levels of homocysteine in the patient's plasma. Long-term determination of the efficacy of the claimed treatment for a particular patient is done by retrospectively evaluating statistics borrowed from hospitals, obstetricians, or pediatricians. Alternatively, after identifying genetic profiles among a number of patients, prospective studies focusing on specific conditions could be conducted to compare health outcomes among pregnant women and children across time.

Potential individuals, who would benefit from the treatment of the novel regimen described above, could be identified by isolating specific genetic SNPs among the group of methylation genes targeted by the Invention and measuring the occurrence of certain conditions. Given large enough studies, data could be collected to identify correlations between specific conditions and single versus combinations of specific SNPs, factoring in whether the SNPs are homozygous or heterozygous.

The studies discussed below use folic acid, B6 (pyridoxal), and B12 (cyanocobalamin) as well as no supplementation to compare with the invention. These supplements are the industry standard forms appearing in the majority of vitamin supplements on the market. Whenever possible, the supplemented dosages of folic acid, pyridoxal and cyanocobalamin should fall within the range of FDA recommended daily allowances. Regarding the invention, the quantities and dosages for research studies adhere to the schedule described herein. In the case of butyrate, where there is no recommended daily allowance, the dosage in the invention is well within the range of quantities safely used in scientific studies of butyrate. In the cases of additional supplements, the dosage in the invention is within range of quantities safely used in scientific studies of said supplements.

Each study is designed to test the efficacy of the invention versus unmodified folate, as well as unmodified folate, B6, and B12 versus a control. All subjects are genotyped for the presence or absence of SNPs in genes of the of the one carbon metabolism pathway (MTHFR, MTR, MTRR, MTHFD1, and SHMT).

Studies related to fertility and assisted reproductive technology were designed with supplementation commencing three months prior to fertilization.

The studies related to prenatal health were designed with supplementation commencing three months prior to conception, as well, in order to optimize egg and sperm quality, and cover the entire nine months of pregnancy.

Studies related to newborn and infant health, in the case of breastmilk feeding, the mothers continued with supplementing. In the case of formula feeding, infants consuming the typical amounts of supplements present in the formula are compared with infants receiving additional supplementation with the invention.

Studies related to toddlers and children, the studies were designed with supplementation commencing at the beginning of the study or up to three months prior.

1) Study to Compare the Effect of Supplementation on the Incidence of the Following Conditions in Pregnant and/or Postpartum Women:

Pre-gestational diabetes
Hyperemesis gravidarum

### Preeclampsia

Spontaneous abortion & miscarriage
Placental abruption
Placental previa
Premature birth
Prolonged or difficult labor
Induced birth

### C-Section

Peri partum depression

### Anemia

Anemia with erythropoietin deficiency
Sickle cell symptoms & complications
After obtaining genetic profiles, compare incidence of any or all of the above conditions among the following groups to see the effects of supplementation:

| Genotype | Supplementation |
|---|---|
| Control (no methylation SNPs) | No supplements |
| Mothers (with SNPs) | No supplements |
| Mothers (with SNPs) | Folic acid |
| Mothers (with SNPs) | Folic acid, B6, B12 |
| Mothers (with SNPs) | 5 MTH folate, methylcobalamin, |
| | pyridoxal-5-phosphate, butyrate |

2) Study to Compare the Effect of Supplementation on the Incidence of the Following Conditions in Newborn to 1-Year Old Babies:

Low Apgar scores
Low birth weight
Premature birth
Congenital malformations and deformations of the
Tongue, mouth, pharynx, including cleft lip & palate, ankyloglossia, macroglossia, hypoglossia
Esophagus & stomach, including esophageal atresia, tracheoesophageal fistula, esophageal web & Schatzki ring, pyloric stenosis, hiatus hernia
Intestines, including intestinal atresia, duodenal atresia, Merckel's diverticulum, Hirschsprung's disease, intestinal malrotation, dolichocolon, enteric duplication cyst
Rectum and anal canal, including imperforate anus, rectovestibular fistula, persistent cloaca, rectal atresia
Pancreas, including annular pancreas, accessory pancreas, Johanson-Blizzzard syndrome, pancreas divisum
Bile ducts and liver, including choledochal cysts, Caroli disease, biliary atresia, Alagille syndrome, polycystic liver disease
Neural tube, brain and spine, including spina bifida, anencephaly, encephalocele, hydrocephalus, facial clefts
Heart and heart vessels, including tetralogy of Fallot, ventricular septal defect, atrial septal defect, patent ductus arteriosus
Renal and urinary tract, including hypospadias, double ureter, obstructive defects of the renal pelvis, renal agenesis, renal dysplasia
Bones, including facial asymmetry, congenital limb amputations, limb hypoplasia, limb deficiencies
Conjoined twins, monozygotic twin loss
Structural or functional defects in the developing embryo or fetus from maternal exposure to environmental toxins
Low concentrations of oxygen and nutrients in the blood
Residual neurological damage from neonatal hypoxic/ischemic injury
Residual damage from prenatal infectious or inflammatory insults
Prenatal immune activation

### Failure to Thrive & Failure to Thrive Syndrome

Delayed closure of fontanelles
Infant eczema
A. After obtaining genetic profiles, compare the incidence of any or all of the above conditions among the following groups to see the effects of prenatal supplementation by their mothers:

| Genotype | Supplementation |
|---|---|
| Control: Mothers, babies (no SNPs) | No supplements |
| Mothers, babies (both with SNPs) | No supplements |
| Mothers (no SNPs), babies (with SNPs) | Folic acid |
| Mothers, babies (both with SNPs) | Folic acid |
| Mothers (no SNPs), babies (with SNPs) | Folic Acid, B6, B12 |
| Mothers, babies (both with SNPs) | Folic Acid, B6, B12 |
| Mothers (no SNPs), babies (with SNPs) | 5MTH folate, methylcobalamin, |
| | pyridoxal-5 phosphate, butyrate |
| Mothers, babies (both with SNPs) | 5MTH folate, methylcobalamin, |
| | pyridoxal-5-phosphate, butyrate |

B. After obtaining genetic profiles, compare the incidence of any or all of the above conditions in infants fed with breastmilk either whose mothers continue supplementing themselves or who provide supplements to the infants directly.

| Genotype | Supplementation |
|---|---|
| Control Breastmilk fed: Mothers, babies (no SNPs) | No supplementation |
| Breastmilk fed: Mothers, babies (both with SNPs) | No supplementation |
| Breastmilk fed: Mothers (no SNPs), babies (with SNPs) | Folic Acid |
| Breastmilk fed: Mothers, babies (both with SNPs) | Folic Acid |
| Breastmilk fed: Mothers (no SNPs), babies (with SNPs) | Folic Acid, B6, B12 |
| Breastmilk fed: Mothers, babies (both with SNPs) | Folic Acid, B6, B12 |
| Breastmilk fed: Mothers (no SNPs), babies (with SNPs) | 5MTH folate, methylcobalamin, |
| | pyridoxal-5-phosphate, butyrate |
| Breastmilk fed: Mothers (no SNPs), babies (with SNPs) | 5MTH folate, methylcobalamin, |
| | pyridoxal-5-phosphate, butyrate |

C. After obtaining genetic profiles, compare the incidence of any or all of the above conditions in infants fed with standardized infant formula versus formula to which the Invention has been added:

| Genotype | Supplementation |
|---|---|
| Control Infants (with no SNPs) | No supplements |
| Infants (with SNPs) | No supplements |
| Infants (with SNPS) | Folic acid |
| Infants (with SNPs) | Folic acid, B6, B12 |
| Infants (with SNPs) | 5 MTH folate, methylcobalamin, |
| | pyridoxal-5-phosphate, butyrate |

3) Study to Compare the Effect of Supplementation on the Incidence of the Following Conditions in 1 to 3-Year Old Toddlers:

### Failure to Thrive & Failure to Thrive Syndrome

Delayed closure or incomplete ossification of the fontanelles
Delayed, asymmetrical, underdeveloped facial bones, including jaws and teeth
Delayed speech, language acquisition

### Pervasive Developmental Disorder (PDD)

Separation Trauma in premature infants, and hospitalized neonates, infants and preschoolers
Intestinal inflammation, leaky gut, loss of integrity of cellular tight junctions
Celiac, gluten, gliadin sensitivities

### Allergies

### Anemia

Anemia with erythropoietin deficiency
Infant eczema, atopic dermatitis
Environmental toxin damage to lungs and bronchial tissues, organs, brain, skin
Neurological damage resulting from immune activation, including high fevers, febrile seizures, bacterial & viral infections
Growth and mental retardation resulting from low concentrations of oxygen and nutrients in the blood
Delayed repair of injuries to the bones and/or epiphyseal plates
Vaccination toxicity, hyper immune response to vaccinations
After obtaining genetic profiles, compare incidence of any or all of the above conditions among the following groups to see the effects of supplementation:

| Genotype | Supplementation |
|---|---|
| Control (no methylation SNPs) | No supplements |
| Infants, toddlers (with SNPs) | No supplements |
| Infants, toddlers (with SNPS) | Folic acid |
| Infants, toddlers (with SNPs) | Folic acid, B6, B12 |
| Infants, toddlers (with SNPs) | 5 MTH folate, methylcobalamin, |
| | pyridoxal-5-phosphate, butyrate |

4) Study to Compare the Effect of Supplementation on the Incidence of the Following Conditions in 4 to 12-Year Old Children:

Delayed, asymmetrical, underdeveloped facial bones, including jaws and teeth
Decelerated or arrested physical growth
Delayed repair of injuries to the bones and/or epiphyseal plates
Delayed speech, language acquisition

### Pervasive Developmental Disorder (PDD)

Intestinal inflammation, leaky gut, loss of integrity of cellular tight junctions
Celiac, gluten, gliadin sensitivities

### Allergies

### Anemia

Anemia with erythropoietin deficiency
Sickle cell symptoms and complications
Eczema, atopic dermatitis
Generalized anxiety and panic disorders, Obsessive Compulsive Disorder (OCD), Attention Deficit Disorder (ADD), Attention Deficit Hyperactivity Disorder (ADHD), Deficits in Attention, Motor control and Perception (DAMP)
Autism Spectrum Disorders (ASD), Delayed Neurological Development Syndromes, Pediatric Autoimmune Neuropsychiatric Disorders Associated with Streptococcal Infections (PANDAS) Environmental toxin damage to lungs and bronchial tissues, organs, brain, skin
Neurological damage resulting from immune activation, including high fevers, febrile seizures, bacterial & viral infections
Growth and mental retardation resulting from low concentrations of oxygen and nutrients in the blood
Vaccination toxicity, hyper immune response to vaccinations
After obtaining genetic profiles, compare incidence of any or all of the above conditions among the following groups to see the effects of supplementation:

| Genotype | Supplementation |
|---|---|
| Control (no methylation SNPs) | No supplements |
| 4-12 year olds (with SNPs) | No supplements |
| 4-12 year olds (with SNPs) | Folic acid |
| 4-12 year olds (with SNPs) | Folic Acid, B6, B12 |
| 4-12 year olds (with SNPs) | 5 MTH folate, methylcobalamin, |
| | pyridoxal-5-phosphate, butyrate |

5) Studies to Compare the Effect of Minimum 3 Months Prior Supplementation on the Incidence of the Following Conditions Related to Fertility and Prospective Parents Using ART:

Low Sperm Count, Low Motility and Abnormal Morphology:

After obtaining genetic profiles, compare incidence of the above conditions among the following groups of men:

| Genotype | Supplementation |
|---|---|
| Control (men with no SNPs) | No supplements |
| Men (with SNPs) | No supplements (men with SNPs) |
| Men (with SNPs) | Folic acid |
| Men (with SNPs) | Folic acid, B6, B12 |
| Men (with SNPs) | 5 MTH folate, methylcobalamin, |
| | pyridoxal-5-phosphate, butyrate |

Implantation Rate of Embryos in ART:

After obtaining genetic profiles, compare incidence of the implantation rate among the following groups of women:

| Genotype | Supplementation |
|---|---|
| Control (women with no SNPs): | No supplements |
| Women (with SNPs) | No supplements |
| Women (with SNPs) | Folic acid |
| Women (with SNPs) | Folic acid, B6, B12 |
| Women (with SNPs) | 5 MTH folate, methylcobalamin, |
| | pyridoxal-5-phosphate, butyrate |

Advanced Embryo Selection for ART Using Array CGH:

After obtaining genetic profiles, compare incidence of incorrect chromosome count in embryos from the following groups:

| Genotype | Supplementation |
|---|---|
| Control (parents with no SNPs) | No supplements |
| One Parent only (with SNPs) | No supplements |
| Both Parents (with SNPs) | No supplements |
| One Parent only (with SNPs) | Folic acid |
| Both Parents (with SNPs) | Folic acid |
| One parent only (with SNPs) | Folic acid, B6, B12 |
| Both Parents (with SNPS) | Folic acid, B6, B12 |
| One parent only (with SNPs) | 5MTH Folate, methylcobalamin, |
| | pyridoxal-5-phosphate, butyrate |
| Both Parents (with SNPs) | 5MTH Folate, methylcobalamin, |
| | pyridoxal-5-phosphate, butyrate |

Fertilization Failure of the Human Egg in ART:

After obtaining genetic profiles, compare the incidence of fertilization among the following groups:

| Genotype | Supplementation |
|---|---|
| Control (parents with no SNPs) | No supplements |
| One parent only (with SNPs) | No supplements |
| Both Parents (with SNPs) | No supplements |
| One parent only (with SNPs) | Folic acid |
| Both Parents (with SNPs) | Folic acid |
| One parent only (with SNPs) | Folic acid, B6, B12 |
| Both Parents (with SNPs) | Folic acid, B6, B12 |
| One parent only (with SNPs) | 5MTH Folate, methylcobalamin, |
| | pyridoxal-5-phosphate, butyrate |
| Both Parents (with SNPs) | 5MTH Folate, methylcobalamin, |
| | pyridoxal-5-phosphate, butyrate |

6) Short Term Study to Compare the Effect of Supplementation by the Mother During Pregnancy on the Incidence of the Following Conditions in Infants:

Low apgar scores
Low birth weight
Decelerated physical growth
Neural tube, brain and spine defects
Structural or functional defects in the developing embryo or fetus from maternal exposure to
environmental toxins
Low concentrations of oxygen and nutrients in the blood
Neurological dysfunction resulting from prenatal hypoxia
Pre and perinatal immune activation
Increase in methylation SNPs
The mothers of the infants in this study are inhabitants of a common neighborhood or share a common workplace where unacceptable levels of pollutants have been identified.
After obtaining genetic profiles, compare incidence of any or all of the above conditions among the following groups to see the effects of supplementation:

| Genotype | Supplementation |
|---|---|
| Control (no methylation SNPs) | No supplements |
| Mothers (with SNPs) | No supplements |
| Mothers (with SNPs) | Folic acid |
| Mothers (with SNPs) | Folic acid, B6, B12 |
| Mothers (with SNPs) | 5 MTH folate, methylcobalamin, |
| | pyridoxal-5-phosphate, butyrate |
| Mothers (with no SNPs) | Epifolin and Epifolin+ with |
| | the addition of one or more of |
| | the following: vitamin D3 |
| | (cholecalciferol), selenium, |
| | zinc, potassium citrate, |
| | magnesium citrate. manganese |
| | gluconate dihydrate, lutein, |
| | quercetin dihydrate or other |
| | dietary flavonoid, curcumin, |
| | turmeric (curcuma longa), |
| | ashwaganda (Withania somnifera), |
| | milk thistle, the steroidal |
| | saponins, triterpenoid saponins |
| | and/or the biologically active |
| | compounds in these herbs. |
| Mothers (with SNPs) | Epifolin and Epifolin+ with |
| | the addition of one or more of |
| | the following: vitamin D3 |
| | (cholecalciferol), selenium, |
| | zinc, potassium citrate, |
| | magnesium citrate. manganese |
| | gluconate dihydrate, lutein, |
| | quercetin dihydrate or other |
| | dietary flavonoid, curcumin, |
| | turmeric (curcuma longa), |
| | ashwaganda (Withania somnifera), |
| | milk thistle, the steroidal |
| | saponins, triterpenoid saponins |
| | and/or the biologically active |
| | compounds in these herbs. |

7) Short Term Study to Compare the Effect of Supplementation on the Incidence of the Following Conditions in 1 to 12-Year Old Children:

Frequency of asthma attacks
Shortness of breath
Cardiac arrhythmia

### Fatigue

Headaches and anxiety
Developmental delay including height and weight
Neurodevelopmental disorders
increase in methylation SNPs
The subjects in the studies will be specific age cohorts of inhabitants of a certain neighborhood, attend a particular day care center, preschool or school where there are measurable levels of pollutants, etc.
After obtaining genetic profiles, compare incidence of any or all of the above conditions among the following groups to see the effects of supplementation:

| Genotype | Supplementation |
|---|---|
| Control (no methylation SNPs) | No supplements |
| 1-12 year olds (with SNPs) | No supplements |
| 1-12 year olds (with SNPs) | Folic acid |
| 1-12 year olds (with SNPs) | Folic Acid, B6, B12 |
| 1-12 year olds (with SNPs) | 5 MTH folate, methylcobalamin, |
| | pyridoxal-5-phosphate, butyrate |
| 1-12 year olds (with no SNPS) | Epifolin and Epifolin+ with |
| | the addition of one or more of |
| | the following: vitamin D3 |
| | (cholecalciferol), selenium, |
| | zinc, potassium citrate, |
| | magnesium citrate. manganese |
| | gluconate dihydrate, lutein, |
| | quercetin dihydrate or other |
| | dietary flavonoid, curcumin, |
| | turmeric (curcuma longa), |
| | ashwaganda (Withania somnifera), |
| | milk thistle, the steroidal |
| | saponins, triterpenoid saponins |
| | and/or the biologically active |
| | compounds in these herbs. |
| 1-12 year olds (with SNPs) | Epifolin and Epifolin+ with |
| | the addition of one or more of |
| | the following: vitamin D3 |
| | (cholecalciferol), selenium, |
| | zinc, potassium citrate, |
| | magnesium citrate. manganese |
| | gluconate dihydrate, lutein, |
| | quercetin dihydrate or other |
| | dietary flavonoid, curcumin, |
| | turmeric (curcuma longa), |
| | ashwaganda (Withania somnifera), |
| | milk thistle, the steroidal |
| | saponins, triterpenoid saponins |
| | and/or the biologically active |
| | compounds in these herbs. |

8) Long Term Study to Compare the Effect of Supplementation on the Incidence of the Following Conditions in 1 to 12-Year Old Children:

Frequency of asthma attacks
Shortness of breath, cellular damage to lung and bronchial tissues
Cardiac arrhythmia

### Fatigue

Headaches and anxiety
Developmental delay including height and weight
Neurodevelopmental disorders and impaired brain functions
Increase in methylation SNPs
Lung, liver, kidney, spleen damage
Nervous system disorders

### Cancers

Reproductive disorders
Autoimmune disorders
Increase in methylation SNPs
Chromosomal damage
After obtaining genetic profiles, compare incidence of any or all of the above conditions among the following groups to see the effects of supplementation:
The subjects in the studies will be specific age cohorts of inhabitants of a certain neighborhood, attend a particular day care center, preschool or school where there are measurable levels of pollutants, etc.

| Genotype | Supplementation |
|---|---|
| Control (no methylation SNPs) | No supplements |
| 1-12 year olds (with SNPs) | No supplements |
| 1-12 year olds (with SNPs) | Folic acid |
| 1-12 year olds (with SNPs) | Folic Acid, B6, B12 |
| 1-12 year olds (with SNPs) | 5 MTH folate, methylcobalamin, |
| | pyridoxal-5-phosphate, butyrate |
| 1-12 year olds (with no SNPS) | Epifolin and Epifolin+ with |
| | the addition of one or more of |
| | the following: vitamin D3 |
| | (cholecalciferol), selenium, |
| | zinc, potassium citrate, |
| | magnesium citrate. manganese |
| | gluconate dihydrate, lutein, |
| | quercetin dihydrate or other |
| | dietary flavonoid, curcumin, |
| | turmeric (curcuma longa), |
| | ashwaganda (Withania somnifera), |
| | milk thistle, the steroidal |
| | saponins, triterpenoid saponins |
| | and/or the biologically active |
| | compounds in these herbs. |
| 1-12 year olds (with SNPS) | Epifolin and Epifolin+ with |
| | the addition of one or more of |
| | the following: vitamin D3 |
| | (cholecalciferol), selenium, |
| | zinc, potassium citrate, |
| | magnesium citrate. manganese |
| | gluconate dihydrate, lutein, |
| | quercetin dihydrate or other |
| | dietary flavonoid, curcumin, |
| | turmeric (curcuma longa), |
| | ashwaganda (Withania somnifera), |
| | milk thistle, the steroidal |
| | saponins, triterpenoid saponins |
| | and/or the biologically active |
| | compounds in these herbs. |

9) Short Term Study to Compare the Effect of Supplementation by the Mother During Pregnancy on the Following Symptoms in the Mother:

Generalized anxiety and panic disorders
Sense of doom

### Depression

### Headaches

Panic attacks

### Irritability

Pounding heart
Breathing problems
Loss of libido
Upset stomach, loss of appetite
Chronic constipation
After obtaining genetic profiles, compare incidence of any or all of the above conditions among the following groups to see the effects of supplementation:

| Genotype | Supplementation |
|---|---|
| Control (women with no SNPs): | No supplements |
| Women (with SNPs) | No supplements |
| Women (with SNPs) | Folic acid |
| Women (with SNPs) | Folic acid, B6, B12 |
| Women (with SNPs) | 5 MTH folate, methylcobalamin, |
| | pyridoxal-5-phosphate, butyrate |
| Women (with no SNPs) | Epifolin and Epifolin+ with the |
| | addition of one or more of the |
| | following: L-theanine, GABA (gamma- |
| | aminobutyric acid), magnesium |
| | citrate, potassium citrate, |
| | quercetin dihydrate or other |
| | flavonoids, curcumin, turmeric |
| | (curcuma longa), white peony |
| | (Paeoniae lactiflorae radix), |
| | schisandra (Schisandra Chinenesis |
| | fructus), Porea (Poriae Cocos |
| | sclerotium), lily bulb (Lillium |
| | brownii bulb), red jujube dates |
| | (Zizyphi spinosis fructus) zizyphus |
| | (Zizyphi spinosi semen), licorice |
| | (Glycyrrhiza uralensis radix), the |
| | steroidal saponins, the triterpenoid |
| | saponins and/or the biologically |
| | active compounds in these herbs, |
| Women (with SNPs) | Epifolin and Epifolin+ with the |
| | addition of one or more of the |
| | following: L-theanine, GABA (gamma- |
| | aminobutyric acid), magnesium |
| | citrate, potassium citrate, |
| | quercetin dihydrate or other |
| | flavonoids, curcumin, turmeric |
| | (curcuma longa), white peony |
| | (Paeoniae lactiflorae radix), |
| | schisandra (Schisandra Chinenesis |
| | fructus), Porea (Poriae Cocos |
| | sclerotium), lily bulb (Lillium |
| | brownii bulb), red jujube dates |
| | (Zizyphi spinosis fructus) zizyphus |
| | (Zizyphi spinosi semen), licorice |
| | (Glycyrrhiza uralensis radix), the |
| | steroidal saponins, the triterpenoid |
| | saponins and/or the biologically |
| | active compounds in these herbs, |

10) Short Term Study to Compare the Effect of Supplementation by the Mother During Pregnancy on the Following Symptoms in the Infant:

Premature birth
Low birth weight
Decelerated physical growth
Neural tube, brain and spine defects
Structural or functional defects in the developing embryo or fetus from maternal exposure to
environmental toxins
Difficulty breastfeeding, delayed Latch
Infant colic
After obtaining genetic profiles, compare incidence of any or all of the above conditions among the following groups to see the effects of supplementation:

| Genotype | Supplementation |
|---|---|
| Control (no methylation SNPs) | No supplements |
| Mothers (with SNPs) | No supplements |
| Mothers (with SNPs) | Folic acid |
| Mothers (with SNPs) | Folic acid, B6, B12 |
| Mothers (with SNPs) | 5 MTH folate, methylcobalamin, |
| | pyridoxal-5-phosphate, butyrate |
| Mothers (with no SNPs) | Epifolin and Epifolin+ with the |
| | addition of one or more of the |
| | following: L-theanine, GABA (gamma- |
| | aminobutyric acid), magnesium |
| | citrate, potassium citrate, |
| | quercetin dihydrate or other |
| | flavonoids, curcumin, turmeric |
| | (curcuma longa), white peony |
| | (Paeoniae lactiflorae radix), |
| | schisandra (Schisandra Chinenesis |
| | fructus), Porea (Poriae Cocos |
| | sclerotium), lily bulb (Lillium |
| | brownii bulb), red jujube dates |
| | (Zizyphi spinosis fructus) zizyphus |
| | (Zizyphi spinosi semen), licorice |
| | (Glycyrrhiza uralensis radix), the |
| | steroidal saponins, the triterpenoid |
| | saponins and/or the biologically |
| | active compounds in these herbs. |
| Mothers (with SNPs) | Epifolin and Epifolin+ with the |
| | addition of one or more of the |
| | following: L-theanine, GABA (gamma- |
| | aminobutyric acid), magnesium |
| | citrate, potassium citrate, |
| | quercetin dihydrate or other |
| | flavonoids, curcumin, turmeric |
| | (curcuma longa), white peony |
| | (Paeoniae lactiflorae radix), |
| | schisandra (Schisandra Chinenesis |
| | fructus), Porea (Poriae Cocos |
| | sclerotium), lily bulb (Lillium |
| | brownii bulb), red jujube dates |
| | (Zizyphi spinosis fructus) zizyphus |
| | (Zizyphi spinosi semen), licorice |
| | (Glycyrrhiza uralensis radix), the |
| | steroidal saponins, the triterpenoid |
| | saponins and/or the biologically |
| | active compounds in these herbs |

11) Short Term Study to Compare the Effect of Supplementation on the Incidence of the Following Conditions in 1 to 12-Year Old Children:

Generalized anxiety and panic disorders
Sense of doom

### Depression

### Headaches

Panic attacks, night terrors

### Irritability

Pounding heart
Breathing problems
Upset stomach, loss of appetite
Chronic constipation
Frequent colds
After obtaining genetic profiles, compare incidence of any or all of the above conditions among the following groups to see the effects of supplementation:

| Genotype | Supplementation |
|---|---|
| Control (no methylation SNPs) | No supplements |
| 1-12 year olds (with SNPs) | No supplements |
| 1-12 year olds (with SNPs) | Folic acid |
| 1-12 year olds (with SNPs) | Folic Acid, B6, B12 |
| 1-12 year olds (with SNPs) | 5 MTH folate, methylcobalamin, |
| | pyridoxal-5-phosphate, butyrate |
| 1-12 year olds (with no SNPs) | Epifolin and Epifolin+ with the |
| | addition of one or more of the |
| | following: L-theanine, GABA (gamma- |
| | aminobutyric acid), magnesium |
| | citrate, potassium citrate, |
| | xylose, quercetin dihydrate or |
| | other flavonoids, curcumin, |
| | turmeric (curcuma longa), white |
| | peony (Paeoniae lactiflorae radix), |
| | schisandra (Schisandra Chinenesis |
| | fructus), Porea (Poriae Cocos |
| | sclerotium), lily bulb (Lillium |
| | brownii bulb), red jujube dates |
| | (Zizyphi spinosis fructus) zizyphus |
| | (Zizyphi spinosi semen), licorice |
| | (Glycyrrhiza uralensis radix), the |
| | steroidal saponins, the triterpenoid |
| | saponins and/or the biologically |
| | active compounds in these herbs. |
| 1-12 year olds (with SNPs) | Epifolin and Epifolin+ with the |
| | addition of one or more of the |
| | following: L-theanine, GABA (gamma- |
| | aminobutyric acid), magnesium |
| | citrate, potassium citrate, |
| | xylose, quercetin dihydrate or |
| | other flavonoids, curcumin, |
| | turmeric (curcuma longa), white |
| | peony (Paeoniae lactiflorae radix), |
| | schisandra (Schisandra Chinenesis |
| | fructus), Porea (Poriae Cocos |
| | sclerotium), lily bulb (Lillium |
| | brownii bulb), red jujube dates |
| | (Zizyphi spinosis fructus) zizyphus |
| | (Zizyphi spinosi semen), licorice |
| | (Glycyrrhiza uralensis radix), the |
| | steroidal saponins, the triterpenoid |
| | saponins and/or the biologically |
| | active compounds in these herbs. |

12) Short Term Study to Compare the Effect of Supplementation in the Treatment on the Incidence of the Following Conditions in Prospective Fathers:

Low sperm count
Low sperm motility
Abnormal sperm morphology
Increased incidence of chromosomal abnormalities in spermatozoa
After obtaining genetic profiles, compare incidence of any or all of the above conditions among the following groups to see the effects of supplementation:

| Genotype | Supplementation |
|---|---|
| Control (men with no SNPs) | No supplements |
| Men (with SNPs) | No supplements (men with SNPs) |
| Men (with SNPs) | Folic acid |
| Men (with SNPs) | Folic acid, B6, B12 |
| Men (with SNPs) | 5 MTH folate, methylcobalamin, |
| | pyridoxal-5-phosphate, butyrate |
| Men (with no SNPs) | Epifolin and Epifolin+ with the |
| | addition of one or more of the |
| | following: vitamin D3 (cholecalciferol), |
| | selenium, zinc, quercetin dihydrate |
| | or other dietary flavonoid, ashwaganda |
| | (Withania somnifera), polygonum |
| | multiflorum root, eucommia bark, |
| | astragalus root, or epimedium |
| | grandiflorum herba, the steroidal |
| | saponins, triterpenoid saponins, and/or |
| | the biologically active compounds in |
| | these herbs. |
| Men (with SNPs) | Epifolin and Epifolin+ with the |
| | addition of one or more of the |
| | following: vitamin D3 (cholecalciferol), |
| | selenium, zinc, quercetin dihydrate |
| | or other dietary flavonoid, ashwaganda |
| | (Withania somnifera), polygonum |
| | multiflorum root, eucommia bark, |
| | astragalus root, or epimedium |
| | grandiflorum herba, the steroidal |
| | saponins, triterpenoid saponins, and/or |
| | the biologically active compounds in |
| | these herbs. |

13) Short Term Study to Compare the Effect of (Daily?) Supplementation on the Incidence of the Following Conditions in Prospective Mothers:

Infertility related to PCOS
Age related fertility decline
Hormone imbalance, menstrual irregularity
DES syndrome
Failure to implant
Spontaneous abortion, miscarriage
Pre eclampsia and birth complications, including premature birth
Decreased fertility as measured by inability to conceive naturally
Increased incidence of chromosomal abnormalities in oocytes
After obtaining genetic profiles, compare incidence of any or all of the above conditions among the following groups to see the effects of supplementation:

| Genotype | Supplementation |
|---|---|
| Control (women with no SNPs): | No supplements |
| Women (with SNPs) | No supplements |
| Women (with SNPs) | Folic acid |
| Women (with SNPs) | Folic acid, B6, B12 |
| Women (with SNPs) | 5 MTH folate, methylcobalamin, |
| | pyridoxal-5-phosphate, butyrate |
| Women (with no SNPs) | Epifolin and Epifolin+ with the |
| | addition of one or more of the |
| | following: vitamin D3 (cholecalciferol), |
| | choline, myo-inositol, quercetin |
| | dihydrate or other bioflavonoids, |
| | vitex agnus-castus fruit, dong quai |
| | root, rehmannia root, polygonum |
| | multiflorum root, liycium fruit, the |
| | steroidal saponins, triterpenoid |
| | saponins, and/or the biologically |
| | active compounds in these herbs. |
| Women (with SNPs) | Epifolin and Epifolin+ with the |
| | addition of one or more of the |
| | following: vitamin D3 (cholecalciferol), |
| | choline, myo-inositol, quercetin |
| | dihydrate or other bioflavonoids, |
| | vitex agnus-castus fruit, dong quai |
| | root, rehmannia root, polygonum |
| | multiflorum root, liycium fruit, the |
| | steroidal saponins, triterpenoid |
| | saponins, and/or the biologically |
| | active compounds in these herbs. |

14) Short Term Study to Compare the Effect of Supplementation on the Incidence of the Following Conditions in 4 to 12-Year Old Children:

### Attention Deficit Disorder (ADD)

### Attention Deficit Hyperactivity Disorder (ADHD)

### Obsessive Compulsive Disorder (OCD)

### Tourette's Syndrome

### Sensory Processing Disorders

### Learning Disorders

### Autism

### “Brain Fog”

Nervous Tics, repetitive motions
After obtaining genetic profiles, compare incidence of any or all of the above conditions among the following groups to see the effects of supplementation:

| Genotype | Supplementation |
|---|---|
| Control (no methylation SNPs) | No supplements |
| 1-12 year olds (with SNPs) | No supplements |
| 1-12 year olds (with SNPs) | Folic acid |
| 1-12 year olds (with SNPs) | Folic Acid, B6, B12 |
| 1-12 year olds (with SNPs) | 5 MTH folate, methylcobalamin, |
| | pyridoxal-5-phosphate, butyrate |
| 1 -12 year olds (no SNPs) | Epifolin and Epifolin+ with the |
| | addition of one or more of the |
| | following: choline, lutein, |
| | quercetin dihydrate or other |
| | bioflavonoid, zizyphus (zizyphi |
| | spinosi semen), Chinese wolfberry |
| | (fructus lycium), Atractylodis |
| | macrocephalae rhizome, white peony |
| | (Paeoniae lactiflorae radix), |
| | Polygonum multiflorum root, |
| | Eucommia bark, vitex (Vitex agnus- |
| | castus) fruit, Platycodon |
| | grandifloras, the steroidal |
| | saponinds, triterpenoids saponins, |
| | and/or biologically active compounds |
| | in these herbs. |
| 1-12 year olds (with SNPs) | Epifolin and Epifolin+ with the |
| | addition of one or more of the |
| | following: choline, lutein, |
| | quercetin dihydrate or other |
| | bioflavonoid, zizyphus (zizyphi |
| | spinosi semen), Chinese wolfberry |
| | (fructus lycium), Atractylodis |
| | macrocephalae rhizome, white peony |
| | (Paeoniae lactiflorae radix), |
| | Polygonum multiflorum root, |
| | Eucommia bark, vitex (Vitex agnus- |
| | castus) fruit, Platycodon |
| | grandifloras, the steroidal |
| | saponinds, triterpenoids saponins, |
| | and/or biologically active compounds |
| | in these herbs. |

15) Short Term Study to Compare the Effect of Supplementation on the Incidence of the Following Conditions Post Vaccination in Infants to 12-Year Old Children:

Local reactions with swelling, tenderness, redness lasting up to 7 days

### Fever

Delayed reactions usually fever and rash 1 to 2 weeks later
After obtaining genetic profiles, compare incidence of any or all of the above conditions among the following groups to see the effects of supplementation for one day prior to, the day of, and two days after vaccination:

| Genotype | Supplementation |
|---|---|
| Control (no methylation SNPs) | No supplements |
| 1-12 year olds (with SNPs) | No supplements |
| 1-12 year olds (with SNPs) | Folic acid |
| 1-12 year olds (with SNPs) | Folic Acid, B6, B12 |
| 1-12 year olds (with SNPs) | 5 MTH folate, methylcobalamin, |
| | pyridoxal-5-phosphate, butyrate |
| 1 -12 year olds (no SNPs) | Epifolin and Epifolin+ with the |
| | addition of one or more of the |
| | following: potassium citrate, |
| | magnesium citrate |
| 1-12 year olds (with SNPs) | Epifolin and Epifolin+ with the |
| | addition of one or more of the |
| | following: potassium citrate, |
| | magnesium citrate |

### Long Term Study to Compare the Effect of Supplementation on the Incidence of the Following Conditions Post Vaccination in 1 to 12-Year Old Children:

MMR-related febrile seizures
Rheumatoid arthritis

### Guillaine Barr Syndrome

Thrombocytopenic purpura

### Myopericarditis

### Autism

Multiple sclerosis
After obtaining genetic profiles, compare incidence of any or all of the above conditions among the following groups to see the effects of supplementation for one day prior to, the day of, and two days after vaccination:

| Genotype | Supplementation |
|---|---|
| Control (no methylation SNPs) | No supplements |
| 1-12 year olds (with SNPs) | No supplements |
| 1-12 year olds (with SNPs) | Folic acid |
| 1-12 year olds (with SNPs) | Folic Acid, B6, B12 |
| 1-12 year olds (with SNPs) | 5 MTH folate, methylcobalamin, |
| | pyridoxal-5-phosphate, butyrate |
| 1 -12 year olds (no SNPs) | Epifolin and Epifolin+ with the |
| | addition of one or more of the |
| | following: potassium citrate, |
| | magnesium citrate |
| 1-12 year olds (with SNPs) | Epifolin and Epifolin+ with the |
| | addition of one or more of the |
| | following: potassium citrate, |
| | magnesium citrate |

Comparable experiments on the efficacy of the invention may also be performed in mice. Mthfr-null mice are viable and born in the expected Mendelian ratio but exhibit reduced survival and a range of phenotypes, including slower growth, altered brain histology, and behavioral abnormalities (Yeung K Y. et al., (2017) Cell Rep, 21(7): 1795-1808). Existing research on Mthfr-null mice shows that overdosing of folate in pregnant mice leads to pseudo-MTHFR deficiency and altered methyl metabolism, with embryonic growth delay and short-term memory impairment in offspring (Bahous R H et al., (2017) Hum Mol Genet., 26(5):888-900). These studies corroborate the use of activated forms of folate and other B vitamins present in the invention, along with a lower dosing schedule, to prevent both of these outcomes.

To test the effects of the invention on murine neural structure, Mthfr-null mice are supplemented with either folic acid, a combination of folic acid, B6, B12, or a combination of SMTH folate, methylcobalamin, pyridoxal-5-phosphate, and butyrate (approx. 2 mg/kg, recommended level for rodents) (Reeves P G (1997) J. Nutr., 127:838S-841S). Supplementation begins one week prior to fertilization and continues throughout the course of the experiment.

Evaluation of neurological defects in Mthfr-null mice is performed by immunohistology of brain sections at E10.5, E17.5, and P18, with particular focus on the hippocampus. Behavioral tests for mice are performed based on existing protocols at 21 days, including open field test for anxiety, ladder beam test for measurement of gait, novel object recognition test and Y-maze test for memory (Jadavji N M et al., (2012) Mol Gen Metab, 106:149-159).

What is claimed is:

1. A pharmaceutical formulation comprising 5-methyltetrahydrofolate, methylcobalamin, pyridoxyl-5-phosphate or pharmaceutically acceptable salts thereof.

2. The formulation according to claim 1 further comprising one or more short chain fatty acids or the conjugate base of one or more fatty acids or pharmaceutically acceptable salts thereof.

3. The formulation according to claim 2, wherein said conjugate base of a fatty acid is butyrate or a pharmaceutically acceptable salt thereof.

4. The formulation according to either one of claims 1-3, further comprising an excipient.

5. The formulation according to either one of claims 1-3, further comprising a binding agent.

6. The formulation according to either one of claims 1-3, further comprising a surfactant.

7. The formulation according to either one of claims 1-3, further comprising a buffer.

8. The formulation according to claim 4, wherein said excipient is a preservative.

9. The formulation according to claim 8, wherein said preservative is selected from the group consisting of cresols, benzyl alcohol, phenol, benzalkonium chloride, benzethonium chloride, chlorobutanol, phenylethyl alcohol, methyl paraben, propyl paraben, thiomersal, phenylmercuric nitrate and acetate, citric acid, sodium citrate, potassium sorbate, ascorbic acid, sodium benzoate, and vegetable glycerin.

10. The formulation according to either one of claims 1-3, wherein said formulation comprises
between 22 to 150 mcg, preferably 100 mcg, of 5-methyltetrahydrofolate or a pharmaceutically acceptable salt thereof,
between 0.16 to 20 mcg, preferably 0.5 mcg, of methylcobalamin or a pharmaceutically acceptable salt thereof, and
between 0.1 mg to 3.6 mg, preferably 1.8 mg, of pyridoxyl-5-phosphate or a pharmaceutically acceptable salt thereof.

11. The formulation according to either one of claims 1-3, further comprising between 2.0 mg to 200 mg, preferably 100 mg, of butyrate or a pharmaceutically acceptable salt thereof.

12. The formulation according to either one of claims 1-3, wherein said formulation comprises:
between 49 to 300 mcg, preferably 200 mcg, of 5-methyltetrahydrofolate or a pharmaceutically acceptable salt thereof,
between 0.3 to 40 mcg, preferably 1.0 mcg, of methylcobalamin or a pharmaceutically acceptable salt thereof,
between 0.16 mg to 30 mg, preferably 3.6 mg, of pyridoxyl-5-phosphate or a pharmaceutically acceptable salt thereof.

13. The formulation according to claim 12, further comprising between 4.0 mg to 300 mg, preferably 200 mg, of butyrate or a pharmaceutically acceptable salt thereof.

14. The formulation according to either one of claims 1-3, wherein said formulation comprises
between 132 to 600 mcg, preferably 400 mcg, of 5-methyltetrahydrofolate or a pharmaceutically acceptable salt thereof,
between 0.8 to 2,000 mcg, preferably 2.0 mcg, of methylcobalamin or a pharmaceutically acceptable salt thereof,
between 0.56 mg to 40 mg, preferably 7.2 mg, of pyridoxyl-5-phosphate or a pharmaceutically acceptable salt thereof.

15. The formulation according to claim 14, further comprising between 6.0 mg to 500 mg, preferably 400 mg, of butyrate or a pharmaceutically acceptable salt thereof.

16. The formulation according to either one of claims 1-3, wherein said formulation comprises:
between 132 to 1,000 mcg, preferably 400 mcg, of 5-methyltetrahydrofolate or a pharmaceutically acceptable salt thereof,
between 0.8 to 2,000 mcg, preferably 2.4 mcg, of methylcobalamin or a pharmaceutically acceptable salt thereof,
between 0.56 mg to 40 mg, preferably 7.2 mg, of pyridoxyl-5-phosphate or a pharmaceutically acceptable salt thereof.

17. The formulation according to claim 16, further comprising between 10.0 mg to 750 mg, preferably 400 mg, of butyrate or a pharmaceutically acceptable salt thereof.

18. The formulation according to either one of claims 1-3, wherein said formulation comprises:
between 198 to 1,000 mcg, preferably 800 mcg, or methyltetrahydrofolate or a pharmaceutically acceptable salt thereof,
between 0.92 to 10,000 mcg, preferably 350 mcg, of methylcobalamin or a pharmaceutically acceptable salt thereof,
between 0.66 to 100.0 mg, preferably 30.0 mg, of pyridoxyl-5-phosphate or a pharmaceutically acceptable salt thereof.

19. The formulation according to claim 18, further comprising between 20.0 mg to 1,200 mg, preferably 200.0 mg, of butyrate or a pharmaceutically acceptable salt thereof.

20. The formulation according to either one of claims 1-3, wherein said formulation comprises a powder, a capsule, a pill, an elixir, an injection, a suppository or a solution administered intravenously.

21. The formulation according to any of the above claims, wherein said formulation is administered as a food additive or dietary supplement.

22. The formulation according to any of the above claims, further comprising one or more of the following: vitamin D3 (cholecalciferol), selenium, zinc, manganese gluconate dihydrate, lutein, quercetin dihydrate or other dietary flavonoid, curcumin, ashwaganda (Withania somnifera), milk thistle, potassium citrate, magnesium citrate, L-theanine, GABA (gamma-aminobutyric acid), curcumin, white peony (Paeoniae lactiflorae radix), schisandra (Schisandra Chinenesis fructus), Porea (Poriae Cocos sclerotium), lily bulb (Lillium brownii bulb), red jujube dates (Zizyphi spinosis fructus) zizyphus (Zizyphi spinosi semen), licorice (Glycyrrhiza uralensis radix), ashwaganda (Withania somnifera), Polygonum multiflorum root, Eucommia bark, Astragalus root, Epimedium grandiflorum herba, choline, myo-inositol, vitex agnus-castus fruit, dong quai root, rehmannia root, Polygonum multiflorum root, liycium fruit, Chinese wolfberry (Fructus lycium), Atractylodis macrocephalae rhizome, or Platycodon grandifloras.

23. A method of promoting health and preventing disease in a patient with polymorphisms, comprising administering to the patient a supplement as described in any of the above claims.

24. A method of promoting health and preventing disease in a patient with polymorphisms according to claim 23, wherein said polymorphism effects epigenetic signaling.

25. A method of promoting health and preventing disease in a patient with polymorphisms according to claim 24, wherein said epigenetic signaling is involved in folate metabolism.

26. The method according to claim 23, wherein the patient has a polymorphism in at least one gene selected from the group consisting of MTHFR, MTR, MTRR, MTHFD1, and SHMT.

27. The method according to claims 23-26, wherein the patient is 12 years old or younger.

28. The method according to claims 23-26, wherein patient is a preconception parent, or pregnant or nursing mother.

29. The method according to claim 23, wherein the amounts of the compounds of the administered supplement are:
between 22 to 150 mcg, preferably 100 mcg, of 5-methyltetrahydrofolate or a pharmaceutically acceptable salt thereof,
between 0.16 to 20 mcg, preferably 0.5 mcg, of methylcobalamin or a pharmaceutically acceptable salt thereof,
between 0.1 mg to 3.6 mg, preferably 1.8 mg, of pyridoxyl-5-phosphate or a pharmaceutically acceptable salt thereof, and
between 2.0 mg to 200 mg, preferably 100 mg, of butyrate or a pharmaceutically acceptable salt thereof.

30. The method according to claim 23, wherein the amounts of the compounds of the administered supplement are:
between 49 to 300 mcg, preferably 200 mcg, of 5-methyltetrahydrofolate or a pharmaceutically acceptable salt thereof,
between 0.3 to 40 mcg, preferably 1.0 mcg, of methylcobalamin or a pharmaceutically acceptable salt thereof,
between 0.16 mg to 30 mg, preferably 3.6 mg, of pyridoxyl-5-phosphate or a pharmaceutically acceptable salt thereof, and
between 4.0 mg to 300 mg, preferably 200 mg, of butyrate or a pharmaceutically acceptable salt thereof.

31. The method according to claim 23, wherein the amounts of the compounds of the administered supplement are:
between 132 to 600 mcg, preferably 400 mcg, of 5-methyltetrahydrofolate or a pharmaceutically acceptable salt thereof,
between 0.8 to 2,000 mcg, preferably 2.0 mcg, of methylcobalamin or a pharmaceutically acceptable salt thereof,
between 0.56 mg to 40 mg, preferably 7.2 mg, of pyridoxyl-5-phosphate or a pharmaceutically acceptable salt thereof, and
between 6.0 mg to 500 mg, preferably 400 mg, of butyrate or a pharmaceutically acceptable salt thereof.

32. The method according to claim 23, wherein the amounts of the compounds of the administered supplement are:
between 132 to 1,000 mcg, preferably 400 mcg, of 5-methyltetrahydrofolate or a pharmaceutically acceptable salt thereof,
between 0.8 to 2,000 mcg, preferably 2.4 mcg, of methylcobalamin or a pharmaceutically acceptable salt thereof,
between 0.56 mg to 40 mg, preferably 7.2 mg, of pyridoxyl-5-phosphate or a pharmaceutically acceptable salt thereof, and
between 10.0 mg to 750 mg, preferably 400 mg. of butyrate or a pharmaceutically acceptable salt thereof.

33. The method according to claim 23, wherein the amounts of the compounds of the administered supplement are:
between 198 to 1000 mcg, preferably 800 mcg, of 5-methyltetrahydrofolate or a pharmaceutically acceptable salt thereof,
between 0.92 to 10,000 mcg, preferably 350 mcg, of methylcobalamin or a pharmaceutically acceptable salt thereof,
between 0.66 mg to 100 mg, preferably 30 mg, of pyridoxyl-5-phosphate or a pharmaceutically acceptable salt thereof, and
between 20.0 mg to 1,200 mg, preferably 200 mg of butyrate or a pharmaceutically acceptable salt thereof.

34. The method according to any of claims 23-33, wherein said supplement is administered as a powder, a capsule, a pill, an elixir, an injection, a suppository or intravenously.

35. The method according to any of claims 23-33, wherein said supplement is administered as a food additive or dietary supplement.
